(19) United States
(12) Patent Application Publication
(10) Pub. No.: US 2019/0282827 A1
(43) Pub. Date: Sep. 19, 2019

(54) PAD-FIXED LASER THERAPEUTIC APPARATUS AND LASER EMITTER

(71) Applicant: LECC TECHNOLOGY CO., LTD., Taoyuan City (TW)

(72) Inventor: HSIN-CHIH TUNG, KINMEN COUNTY (TW)

(21) Appl. No.: 16/207,329

(22) Filed: Dec. 3, 2018

(30) Foreign Application Priority Data
Mar. 14, 2018 (TW) 107203285

(51) Int. Cl. A61N 5/06

(52) U.S. Cl. CPC A61N 5/0613; A61N 2005/067; A61N 2005/0626

(57) ABSTRACT

A pad-fixed laser therapeutic apparatus and a laser emitter are provided. The laser therapeutic apparatus includes a power supply unit, a first electrically-conductive attachment unit and a second electrically-conductive attachment unit attached to an organism, a laser emission unit, an open-circuit detection control unit, and a processing unit. The laser emission unit is fixed on the organism through the first electrically-conductive attachment unit and the second electrically-conductive attachment unit. The open-circuit detection control unit is electrically connected to the first and second electrically-conductive attachment units. The processing unit is electrically connected to the power supply unit, the laser emission unit, and the open-circuit detection control unit. When the open-circuit detection control unit detects that a circuit is in an open-circuit state, the laser emission unit stops emitting a laser, to prevent a laser from irradiating the eyes of another person when the laser emission unit falls off.

### CROSS-REFERENCE TO RELATED PATENT APPLICATION

This application claims the benefit of priority to Taiwan Patent Application No. 107203285, filed on Mar. 14, 2018. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### BACKGROUND OF THE DISCLOSURE

### 1. Field of the Disclosure

The present disclosure relates to a laser therapeutic apparatus and a laser emitter, and in particular, to a pad-fixed laser therapeutic apparatus and a laser emitter.

### 2. Description of Related Art

With the advancement of medical research, the application of low-level laser therapy (LLLT) has become widespread and popular. Most conventional laser therapeutic apparatuses are fixed on acupuncture points or affected areas of users by suction, and a laser emitter emits a low energy laser to perform laser treatment at the areas. However, in practice, since the surface of the affected areas are usually uneven, a laser therapeutic apparatus may easily fall off during fixation by suction, which causes inconvenience and results in unsafe situations where an emitted laser may irradiate the eyes.

On the other hand, although products of low frequency wave electric therapy are commercially available, there is no related product that integrates laser therapy and low frequency wave electric therapy. Therefore, in terms of practicability and convenience, the conventional products are still in need of improvement.

### SUMMARY OF THE DISCLOSURE

In response to the above-referenced technical inadequacies, the present disclosure provides a pad-fixed laser therapeutic apparatus and a laser emitter to resolve deficiencies in the prior art.

The present disclosure provides a pad-fixed laser therapeutic apparatus including a power supply unit, a first electrically-conductive attachment unit, a second electrically-conductive attachment unit, a laser emission unit, an open-circuit detection control unit, and a processing unit. The first electrically-conductive attachment unit is attached to a surface of an organism, and the second electrically-conductive attachment unit is attached to the surface of the organism, where the second electrically-conductive attachment unit and the first electrically-conductive attachment unit cooperatively form a circuit through the organism. The laser emission unit is used to emit a laser. The laser emission unit is fixed on the organism through the first electrically-conductive attachment unit and the second electrically-conductive attachment unit, and the laser emitted by the laser emission unit irradiates the organism. The open-circuit detection control unit is electrically connected to the first electrically-conductive attachment unit and the second electrically-conductive attachment unit. The processing unit is electrically connected to the power supply unit, the laser emission unit, and the open-circuit detection control unit. The processing unit uses a power provided by the power supply unit to drive the laser emission unit to emit the laser. When the open-circuit detection control unit detects that the circuit is in an open-circuit state, the laser emission unit stops emitting the laser.

The present disclosure provides a pad-fixed laser therapeutic apparatus including a main control unit and a laser emitter. The main control unit includes a power supply unit, a first processing unit, and a first connection interface unit. The first processing unit is electrically connected to the power supply unit and the first connection interface unit. The laser emitter includes a second connection interface unit, a first electrically-conductive attachment unit, a second electrically-conductive attachment unit, a laser emission unit, an open-circuit detection control unit, and a second processing unit. The second connection interface unit is electrically connected to the first connection interface unit. The first electrically-conductive attachment unit is attached to a surface of an organism. The second electrically-conductive attachment unit is attached to the surface of the organism. The second electrically-conductive attachment unit and the first electrically-conductive attachment unit cooperatively form a circuit through the organism. The laser emission unit is used to emit a laser. The laser emission unit is fixed on the organism through the first electrically-conductive attachment unit and the second electrically-conductive attachment unit, and the laser emitted by the laser emission unit irradiates the organism. The open-circuit detection control unit is electrically connected to the first electrically-conductive attachment unit and the second electrically-conductive attachment unit. The second processing unit is electrically connected to the second connection interface unit, the laser emission unit, and the open-circuit detection control unit. When the open-circuit detection control unit detects that the circuit is in an open-circuit state, the laser emission unit stops emitting the laser.

The present disclosure provides a pad-fixed laser emitter for a laser therapeutic apparatus including a connection interface unit, a first electrically-conductive attachment unit, a second electrically-conductive attachment unit, a laser emission unit, and an open-circuit detection control unit. The connection interface unit is electrically connected to a main control unit of the laser therapeutic apparatus, to receive a power provided by the main control unit and a control signal sent by the main control unit. The first electrically-conductive attachment unit is attached to a surface of an organism. The second electrically-conductive attachment unit is attached to the surface of the organism. The organism and the first electrically-conductive attachment unit cooperatively form a circuit. The laser emission unit is electrically connected to the connection interface unit to emit a laser. The laser emission unit is fixed on the organism through the first electrically-conductive attachment unit and the second electrically-conductive attachment unit, and the laser emitted by the laser emission unit irradiates the organism. The open-circuit detection control unit is electrically connected to the first electrically-conductive attachment unit and the second electrically-conductive attachment unit. When the open-circuit detection control unit detects that the circuit is in an open-circuit state, the laser emission unit stops emitting the laser.

### BRIEF DESCRIPTION OF THE DRAWINGS

FIG. 1 shows a functional block diagram of a laser therapeutic apparatus according to a first embodiment of the present disclosure;

FIG. 2 shows a functional block diagram of a laser therapeutic apparatus according to a second embodiment of the present disclosure;

FIG. 3 shows a schematic diagram of the laser therapeutic apparatus according to the second embodiment of the present disclosure;

FIG. 4 shows a schematic diagram of the laser therapeutic apparatus according to the second embodiment of the present disclosure;

FIG. 5 shows a schematic diagram of a use state of a laser emitter according to a third embodiment of the present disclosure; and

FIG. 6 shows a partial exploded diagram of a laser emitter according to the third embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like

### First Embodiment

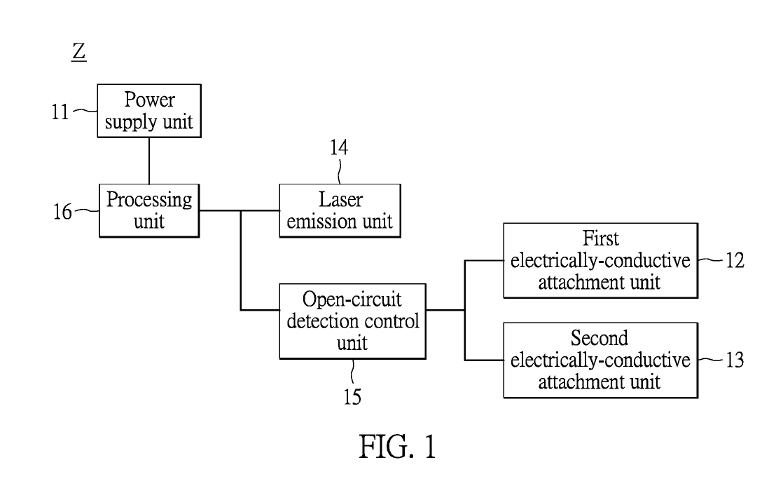

Referring to FIG. 1, a first embodiment of the present disclosure provides a pad-fixed laser therapeutic apparatus Z, including a power supply unit 11, a first electrically-conductive attachment unit 12, a second electrically-conductive attachment unit 13, a laser emission unit 14, an open-circuit detection control unit 15, and a processing unit 16. The processing unit 16 may be, for example, a logic integrated circuit (IC) such as a microcontroller AT89S51, or other electronic elements having a computing function. The processing unit 16 is electrically connected to the power supply unit 11, the laser emission unit 14, and the open-circuit detection control unit 15. The processing unit 16 uses a power provided by the power supply unit 11 to drive the laser emission unit 14 to emit a laser. In practice, a helium-neon laser or a far-infrared laser may be emitted, but the present disclosure is not limited thereto.

In this embodiment, a power of the laser is 5 milliwatts (mW), but the present disclosure is not limited thereto. In practice, the laser emission unit 14 having a power between 2 milliwatts and 8 milliwatts may be used. The power of the laser emission unit 14 is preferably between 3 milliwatts and 6 milliwatts.

The laser emission unit 14 is fixed on an organism through the first electrically-conductive attachment unit 12 and the second electrically-conductive attachment unit 13, and the laser emitted by the laser emission unit 14 irradiates the organism (not shown) to perform low energy laser processing at a position of the organism. Specifically, the first electrically-conductive attachment unit 12 is attached to a surface of the organism, and the second electrically-conductive attachment unit 13 is attached together to the surface of the organism, so that the laser emission unit 14 can be fixed on the organism. The laser emission unit 14 of the present disclosure is fixed on the organism by attachment through the first electrically-conductive attachment unit 12 and the second electrically-conductive attachment unit 13 since the effect of attachment is better than the effect of suction by which the conventional laser electric therapy apparatus is fixed.

Referring to FIG. 1 again, when the first electrically-conductive attachment unit 12 and the second electrically-conductive attachment unit 13 are attached to the organism together, the first electrically-conductive attachment unit 12 and the second electrically-conductive attachment unit 13 can cooperatively form a circuit through the organism. In this embodiment, the open-circuit detection control unit 15 is electrically connected to the first electrically-conductive attachment unit 12 and the second electrically-conductive attachment unit 13 to detect whether the circuit is conductive. When the open-circuit detection control unit 15 detects that the circuit is in an open-circuit state, the laser emission unit 14 stops emitting a laser. Specifically, the open-circuit detection control unit 15 may send a stop signal to the processing unit 16, so that when receiving the stop signal, the processing unit 16 stops driving the laser emission unit 14 to emit a laser. Alternatively, when the circuit is in a conductive state, the open-circuit detection control unit 15 continuously sends an operation signal to the processing unit 16. When the operation signal received by the processing unit 16 is interrupted, the laser emission unit 14 is no longer driven to emit a laser. The present disclosure is not limited to the foregoing examples.

### Second Embodiment

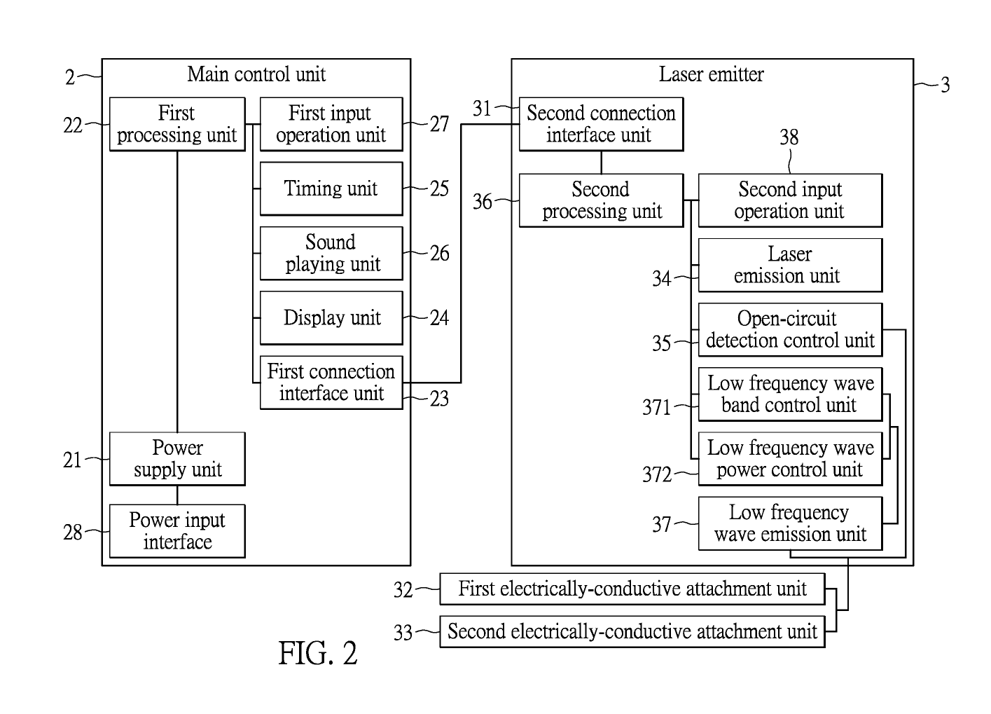

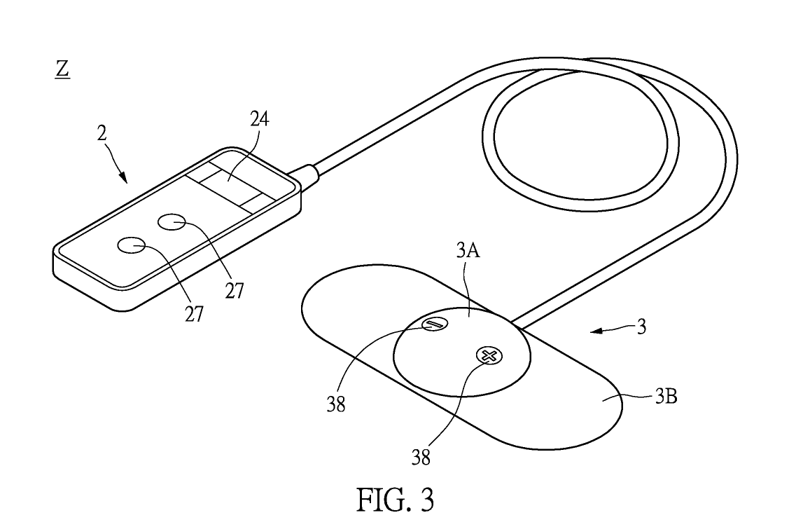

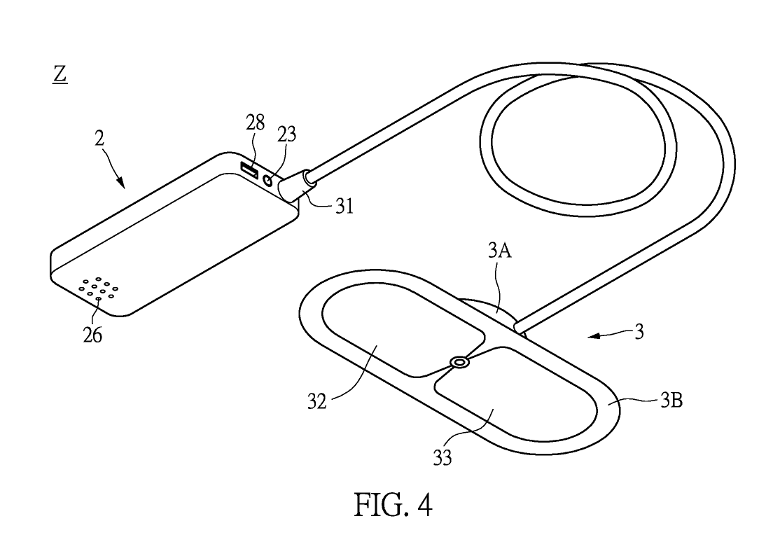

The laser therapeutic apparatus Z in a second embodiment of the present disclosure is described below with reference to FIG. 2 to FIG. 4. Based on the drawings, the second embodiment of the present disclosure provides the pad-fixed laser therapeutic apparatus Z, including a main control unit 2 and a laser emitter 3. The main control unit 2 includes a power supply unit 21, a first processing unit 22, and a first connection interface unit 23. The first processing unit 22 is electrically connected to the power supply unit 21 and the first connection interface unit 23. Accordingly, a power provided by the power supply unit 21 may be transferred to the first connection interface unit 23. The power supply unit 21 may be a battery module installed inside the main control unit 2 and is charged by using a power input interface 28, or may be a battery module replaceably installed inside the main control unit 2, or a power supply connected to mains power, where a transformer is used to convert alternating current (AC) power of the mains power into direct current (DC) power.

The laser emitter 3 includes a second connection interface unit 31, a first electrically-conductive attachment unit 32, a second electrically-conductive attachment unit 33, a laser emission unit 34, an open-circuit detection control unit 35, and a second processing unit 36. The second processing unit 36 is electrically connected to the second connection interface unit 31, the laser emission unit 34, and the open-circuit detection control unit 35.

The laser emitter 3 is electrically connected to the main control unit 2. Specifically, in this embodiment, the second connection interface unit 31 of the laser emitter 3 is electrically connected to the first connection interface unit 23 of the main control unit 2, so that the laser emitter 3 is electrically connected to the main control unit 2. The second processing unit 36 uses the second connection interface unit 31 to receive a power provided by the power supply unit 21 to drive the laser emission unit 34 to emit a laser.

The first electrically-conductive attachment unit 32 and the second electrically-conductive attachment unit 33 are also attached together to cooperatively form a circuit through an organism. The laser emitter 3 is fixed on the organism through the first electrically-conductive attachment unit 32 and the second electrically-conductive attachment unit 33. In this way, the laser emission unit 34 disposed at the laser emitter 3 is fixed on the organism, and the laser emitted by the laser emission unit 34 irradiates the organism.

The open-circuit detection control unit 35 is electrically connected to the first electrically-conductive attachment unit 32 and the second electrically-conductive attachment unit 33. When the open-circuit detection control unit 35 detects that the circuit is in an open-circuit state, the laser emission unit 34 stops emitting a laser.

The main control unit 2 in the second embodiment of the present disclosure is further described below. Referring to FIG. 2 to FIG. 4, the main control unit 2 in the second embodiment of the present disclosure further includes a display unit 24, a timing unit 25, a sound playing unit 26, a first input operation unit 27, and a power input interface 28. The display unit 24, the timing unit 25, the sound playing unit 26 (such as a buzzer), and the first input operation unit 27 are electrically connected to the first processing unit 22.

The display unit 24 is used to display at least one piece of information for a user to understand an operation status of the laser therapeutic apparatus Z, for example, a remaining electric quantity state of the laser therapeutic apparatus Z.

The main control unit 2 in this embodiment further includes the timing unit 25. The main control unit 2 uses the timing unit 25 to perform a timing procedure. For example, if the duration of one course of LLLT is 10 minutes, the timing unit 25 may be used to perform a timing procedure for the duration of 10 minutes. When the timing procedure performed by the timing unit 25 ends, the course of LLLT is ended (the laser emission unit 34 stops emitting a laser). Furthermore, it is possible that after the timing procedure performed by the timing unit 25, the first processing unit 22 drives the sound playing unit 26 to play a sound, so as to remind the user that the course is completed. In another aspect, during the timing procedure performed by the timing unit 25, the display unit 24 may display timing information (for example, countdown seconds, minutes, or the like), so that the user can learn about the progress of the course.

The main control unit 2 further includes the first input operation unit 27. The first input operation unit 27 is a plurality of keys disposed on the main control unit 2. The user may press the keys to perform various input operations. In addition, the first input operation unit 27 may alternatively be a single key, and the user can perform different input operations by pressing the key for different time lengths or by consecutively pressing the key different numbers of times within a short period of time. Alternatively, the first input operation unit 27 is a touch operation panel.

Aside from a power on or off operation, in an embodiment in which the timing unit 25 is disposed, the first input operation unit 27 may be used to set the duration of a course that the user intends to take. After the user operates the first input operation unit 27, the first processing unit 22 receives a signal (for example, a setting signal) sent by the first input operation unit 27 to set a timing procedure to be performed by the timing unit 25. In addition, according to a signal (for example, a start signal) sent by the first input operation unit 27, the first processing unit 22 can drive the timing unit 25 to perform the timing procedure. According to another signal (for example, a pause signal) sent by the first input operation unit 27, the first processing unit 22 can even drive the timing unit 25 to temporarily interrupt the timing procedure, or according to still another signal (for example, a restart signal) sent by the first input operation unit 27, the first processing unit 22 can resume the timing procedure. A power input interface 28 is disposed at the main control unit 2 in this embodiment. The user may use the power input interface 28 to charge the power supply unit 21 installed inside the main control unit 2. The power input interface 28 may be a Micro USB specification connector or a USB Type-C specification connector. However, the present disclosure is not limited thereto. The second connection interface unit 31 of the laser emitter 3 is electrically connected to the first connection interface unit 23 of the main control unit 2, so that the laser emitter 3 is electrically connected to the main control unit 2. Two first connection interface units 23 are disposed on the main control unit 2 so that two laser emitters 3 may be connected to the main control unit 2 at the same time. However, it is possible to dispose merely one first connection interface unit 23 on the main control unit 2, or dispose different numbers of the first connection interface units 23 to correspondingly connect to the different numbers of the laser emitters 3.

The laser emitter 3 is further described below. The laser emitter 3 further includes a low frequency wave emission unit 37, a low frequency wave band control unit 371, a low frequency wave power control unit 372, and a second input operation unit 38. The low frequency wave emission unit 37, the low frequency wave band control unit 371, the low frequency wave power control unit 372, and the second input operation unit 38 are all electrically connected to the second processing unit 36. The low frequency wave emission unit 37 is electrically connected to the second processing unit 36, the first electrically-conductive attachment unit 32, and the second electrically-conductive attachment unit 33. The low frequency wave emission unit 37 emits a low frequency wave to the organism by a combination of the first electrically-conductive attachment unit 32 and the second electrically-conductive attachment unit 33.

The first electrically-conductive attachment unit 32 and the second electrically-conductive attachment unit 33 cooperatively form a circuit through the organism. Therefore, the low frequency wave emission unit 37 may directly use an existing circuit to emit a low frequency wave to the organism to achieve an effect of low frequency wave therapy. Therefore, the effects of LLLT and low frequency wave therapy both can be implemented on one apparatus without an increase in structural complexity, and the user can flexibly choose to turn on either LLLT or low frequency wave therapy or turn on both LLLT and low frequency wave therapy.

The second processing unit 36 is further electrically connected to the low frequency wave band control unit 371 and the low frequency wave power control unit 372. Therefore, the second processing unit 36 can use the low frequency wave band control unit 371 to control a band of the low frequency wave emitted by the low frequency wave emission unit 37, or use the low frequency wave power control unit 372 to control a power of the low frequency wave emitted by the low frequency wave emission unit 37. The second input operation unit 38 is further disposed at the laser emitter 3, so that the user can perform an input operation. The second input operation unit 38 in this embodiment is a plurality of keys disposed at the laser emitter 3. One of the keys is marked with a plus symbol and one of the keys is marked with a minus symbol. When the laser therapeutic apparatus Z is turned on, the low frequency wave emission unit 37 does not actively emit a low frequency wave. When the user presses the second input operation unit 38 marked with the plus symbol, in this case, the second processing unit 36 receives a signal sent by the second input operation unit 38, and uses the low frequency wave power control unit 372 to increase a power of the low frequency wave emitted by the low frequency wave emission unit 37, to perform low frequency wave therapy, and the same occurs when the second input operation unit 38 marked with the minus symbol is pressed. However, the second input operation unit 38 may also be not additionally disposed and only the first input operation unit 27 on the main control unit 2 would be used for controlling. Similarly, the second processing unit 36 may also be not additionally disposed and all signals are transmitted back to the main control unit 2 to be computed by the first processing unit 22 of the main control unit 2.

Lasers having different wavelengths and powers may be provided by using different laser emission units 34. Specifically, a wavelength range of lasers that are usually used is approximately between 405 nm and 980 nm. A laser having a wavelength of 830 nm about (for example, within a range between 820 nm and 840 nm) may be used to perform laser processing on deeper tissues. A laser having a wavelength of 630 nm to 650 nm and power of 3 milliwatts to 4 milliwatts is more suitable for performing laser processing on a body surface. A laser having power of 10 milliwatts to 25 milliwatts may be used to perform epilation. Since the main control unit 2 may be used with different laser emitters 3, with the purchase of one main control unit 2 and different laser emitters 3, the user can perform LLLT by using lasers having different wavelengths and powers according to requirements.

### Third Embodiment

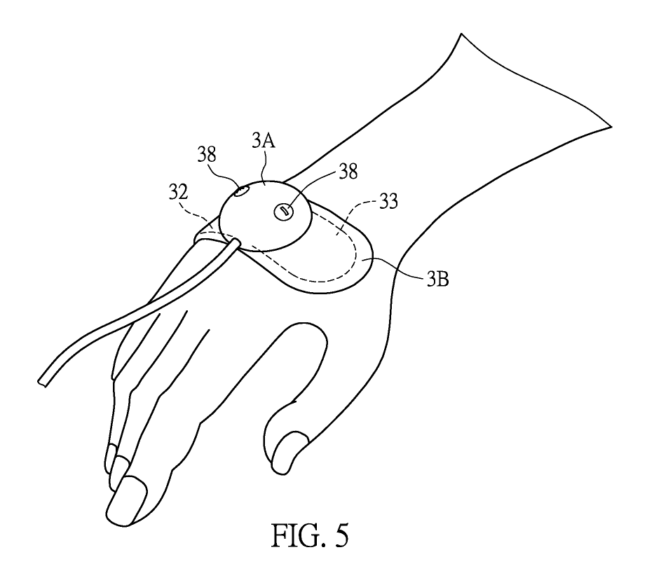

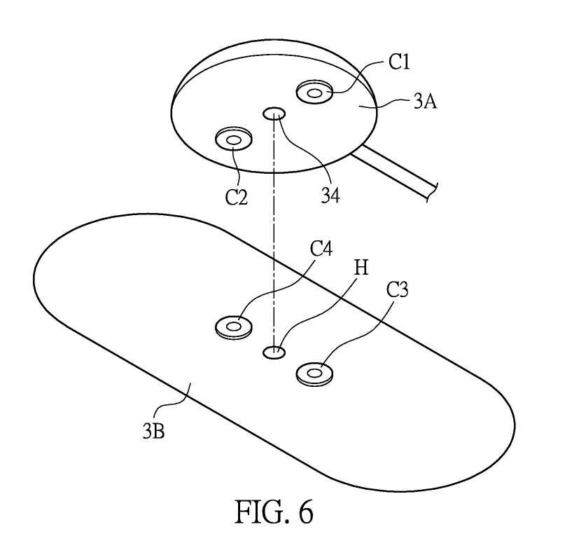

A pad-fixed laser emitter 3 in a third embodiment of the present disclosure is described below by using FIG. 5 and FIG. 6. The third embodiment of the present disclosure provides the pad-fixed laser emitter 3, which is fixed on an organism through a first electrically-conductive attachment unit 32 and a second electrically-conductive attachment unit 33.

The laser emitter 3 in this embodiment is applied to the laser therapeutic apparatus Z. The laser emitter 3 includes a connection interface unit (the foregoing second connection interface unit 31), the first electrically-conductive attachment unit 32, the second electrically-conductive attachment unit 33, the laser emission unit 34, and the open-circuit detection control unit 35 (referring to FIG. 2). Specifically, the laser emitter 3 is electrically connected to the main control unit 2 of the laser therapeutic apparatus Z through the connection interface unit to receive a power provided by the main control unit 2 and a control signal sent by the main control unit 2. After the laser emitter 3 receives the power provided by the main control unit 2 and the control signal, the laser emission unit 34 emits a laser irradiating to the organism.

Referring to FIG. 5 and FIG. 6 again, in this embodiment, the laser emitter 3 includes an emitting head 3A and a bearing sheet body 3B.

The connection interface unit, the laser emission unit 34, and the open-circuit detection control unit 35 are all disposed in the emitting head 3A. In addition, a first electrically-conductive connecting member C1 and a second electrically-conductive connecting member C2 are further disposed at the emitting head 3A. The first electrically-conductive connecting member C1 and the second electrically-conductive connecting member C2 are electrically connected to the open-circuit detection control unit 35.

The first electrically-conductive attachment unit 32 and the second electrically-conductive attachment unit 33 are disposed on the bearing sheet body 3B. The bearing sheet body 3B is a sheet material that has flexibility. The first electrically-conductive attachment unit 32 and the second electrically-conductive attachment unit 33 are disposed on a side of the bearing sheet body 3B. When the bearing sheet body 3B is attached to a surface of an object through the first electrically-conductive attachment unit 32 and the second electrically-conductive attachment unit 33, the bearing sheet body 3B and the first electrically-conductive attachment unit 32 and the second electrically-conductive attachment unit 33 that are on the bearing sheet body 3B may be bent to fit the shape of the surface of the object, so that the bearing sheet body 3B is tightly and stably attached to the surface of the object. A third electrically-conductive connecting member C3 and a fourth electrically-conductive connecting member C4 are disposed on the other side of the bearing sheet body 3B. The third electrically-conductive connecting member C3 is electrically connected to the first electrically-conductive attachment unit 32. The fourth electrically-conductive connecting member C4 is electrically connected to the second electrically-conductive attachment unit 33.

Referring to FIG. 6, the third electrically-conductive connecting member C3 is detachably connected to the first electrically-conductive connecting member C1. The fourth electrically-conductive connecting member C4 is detachably connected to the second electrically-conductive connecting member C2. Accordingly, the first electrically-conductive attachment unit 32 can be electrically connected to the open-circuit detection control unit 35 through the third electrically-conductive connecting member C3 and the first electrically-conductive connecting member C1, and the second electrically-conductive attachment unit 33 can be electrically connected to the open-circuit detection control unit 35 through the fourth electrically-conductive connecting member C4 and the second electrically-conductive connecting member C2. In a preferred embodiment of the present disclosure, the first electrically-conductive connecting member C1, the second electrically-conductive connecting member C2, the third electrically-conductive connecting member C3, and the fourth electrically-conductive connecting member C4 are magnetic-attraction connection fasteners. When the bearing sheet body 3B is close to the emitting head 3A, the magnetic-attraction connection fasteners may be attracted to each other, so that the bearing sheet body 3B can be fixed to the emitting head 3A. In the present disclosure, the electrically-conductive connecting members may be plug-type metal connecting members, or may be metal hidden fasteners.

The bearing sheet body 3B is detachably fixed to the emitting head 3A. Therefore, if the first electrically-conductive attachment unit 32 and the second electrically-conductive attachment unit 33 are contaminated after use or if the first electrically-conductive attachment unit 32 and the second electrically-conductive attachment unit 33 need to avoid being sharing with others for personal hygiene, only the bearing sheet body 3B has to be replaced, so that costs of purchasing related equipment are greatly reduced.

Referring to FIG. 6 again, an opening H is provided on the bearing sheet body 3B. When the bearing sheet body 3B is fixed to the emitting head 3A, the laser emission unit 34 of the emitting head 3A corresponds to the opening H. Accordingly, the laser emitted by the laser emission unit 34 can pass through the opening H to irradiate the organism.

If the emitting head 3A is pulled by an external force during LLLT and falls off from the bearing sheet body 3B adhered to the organism, the circuit among the electrically-conductive connecting members is in an open-circuit state, and therefore the open-circuit detection control unit 35 detects that the circuit is in an open-circuit state and enables the laser emission unit 34 to stop emitting a laser. Accordingly, the laser emitted by the laser emission unit 34 can be prevented from irradiating the eyes of the user or people nearby. In addition, if the user mistakenly pulls off the bearing sheet body 3B during LLLT, the first electrically-conductive attachment unit 32 or the second electrically-conductive attachment unit 33 would be detached from a body surface, and therefore the circuit would be also in an open-circuit state. In this case, the laser emission unit 34 also stops emitting a laser, to avoid the aforementioned situation.

[Beneficial Effects of the Embodiments]

A beneficial effect of the present disclosure lies in that, with the technical solutions that “the laser emission unit 34 is fixed on the organism through the first electrically-conductive attachment unit 32 and the second electrically-conductive attachment unit 33” and “when the open-circuit detection control unit 35 detects that the circuit is in an open-circuit state, the laser emission unit 34 stops emitting a laser,” the pad-fixed laser therapeutic apparatus and the laser emitter provided in the present disclosure achieve both the effects that “the laser emission unit 34 can be stably fixed” and “the laser emitted by the laser emission unit 34 can be prevented from irradiating the eyes of people nearby”.

Furthermore, since the low frequency wave emission unit 37 may directly use an existing circuit. Therefore, the required costs can be effectively reduced and an increase in the structural complexity would not be caused, and even, both the effects of LLLT and low frequency wave therapy can be implemented on the same apparatus.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A pad-fixed laser therapeutic apparatus, comprising:
a power supply unit;
a first electrically-conductive attachment unit, attached to a surface of an organism;
a second electrically-conductive attachment unit, attached to the surface of the organism, wherein the second electrically-conductive attachment unit and the first electrically-conductive attachment unit cooperatively form a circuit through the organism;
a laser emission unit, used to emit a laser, wherein the laser emission unit is fixed on the organism through the first electrically-conductive attachment unit and the second electrically-conductive attachment unit, and the laser emitted by the laser emission unit irradiates the organism;
an open-circuit detection control unit, electrically connected to the first electrically-conductive attachment unit and the second electrically-conductive attachment unit; and
a processing unit, electrically connected to the power supply unit, the laser emission unit, and the open-circuit detection control unit, wherein the processing unit uses a power provided by the power supply unit to drive the laser emission unit to emit the laser, wherein
when the open-circuit detection control unit detects that the circuit is in an open-circuit state, the laser emission unit stops emitting the laser.

2. The laser therapeutic apparatus according to claim 1, wherein a power of the laser is between 2 milliwatts and 8 milliwatts.

3. The laser therapeutic apparatus according to claim 1, further comprising:
a low frequency wave emission unit, electrically connected to the first electrically-conductive attachment unit and the second electrically-conductive attachment unit, wherein the low frequency wave emission unit emits a low frequency wave to the organism by a combination of the first electrically-conductive attachment unit and the second electrically-conductive attachment unit;
a low frequency wave band control unit, electrically connected to the processing unit and the low frequency wave emission unit; and
a low frequency wave power control unit, electrically connected to the processing unit and the low frequency wave emission unit.

4. The laser therapeutic apparatus according to claim 1, further comprising:
a timing unit, electrically connected to the processing unit;
an input operation unit, electrically connected to the processing unit;
a display unit, electrically connected to the processing unit; and
a sound playing unit, electrically connected to the processing unit.

5. A pad-fixed laser therapeutic apparatus, comprising:
a main control unit, including:
a power supply unit;
a first processing unit, electrically connected to the power supply unit; and
a first connection interface unit, electrically connected to the first processing unit; and
a laser emitter, including:
a second connection interface unit, electrically connected to the first connection interface unit;
a first electrically-conductive attachment unit, attached to a surface of an organism;
a second electrically-conductive attachment unit, attached to the surface of the organism, wherein the second electrically-conductive attachment unit and the first electrically-conductive attachment unit cooperatively form a circuit through the organism;
a laser emission unit, used to emit a laser, wherein the laser emission unit is fixed on the organism through the first electrically-conductive attachment unit and the second electrically-conductive attachment unit, and the laser emitted by the laser emission unit irradiates the organism;
an open-circuit detection control unit, electrically connected to the first electrically-conductive attachment unit and the second electrically-conductive attachment unit; and
a second processing unit, electrically connected to the second connection interface unit, the laser emission unit, and the open-circuit detection control unit, wherein the second processing unit uses the second connection interface unit to receive a power provided by the power supply unit to drive the laser emission unit to emit the laser, wherein
when the open-circuit detection control unit detects that the circuit is in an open-circuit state, the laser emission unit stops emitting the laser.

6. The laser therapeutic apparatus according to claim 5, wherein the main control unit further includes:
a timing unit, electrically connected to the first processing unit;
a first input operation unit, electrically connected to the first processing unit;
a display unit, electrically connected to the first processing unit; and
a sound playing unit, electrically connected to the first processing unit, wherein
the first processing unit receives a signal emitted by the first input operation unit, to drive the timing unit to perform a timing procedure,
the first processing unit drives the display unit to display at least one piece of electric quantity information, timing information, and operation information, and
after the timing unit executes the timing procedure, the first processing unit drives the sound playing unit to play a sound.

7. The laser therapeutic apparatus according to claim 5, further comprising:
a low frequency wave emission unit, electrically connected to the second processing unit, the first electrically-conductive attachment unit, and the second electrically-conductive attachment unit, and the low frequency wave emission unit emits a low frequency wave to the organism by a combination of the first electrically-conductive attachment unit and the second electrically-conductive attachment unit;
a low frequency wave band control unit, electrically connected to the second processing unit and the low frequency wave emission unit;
a low frequency wave power control unit, electrically connected to the second processing unit and the low frequency wave emission unit; and
a second input operation unit, electrically connected to the second processing unit, wherein
the second processing unit receives a signal sent by the second input operation unit, to use the low frequency wave band control unit to control a band of the low frequency wave emitted by the low frequency wave emission unit, and
the second processing unit receives a signal sent by the second input operation unit, to use the low frequency wave power control unit to control a power of the low frequency wave emitted by the low frequency wave emission unit.

8. A pad-fixed laser emitter for a laser therapeutic apparatus, comprising:
a connection interface unit, electrically connected to a main control unit of the laser therapeutic apparatus, to receive a power provided by the main control unit and a control signal sent by the main control unit;
a first electrically-conductive attachment unit, attached to a surface of an organism;
a second electrically-conductive attachment unit, attached to the surface of the organism, wherein the organism and the first electrically-conductive attachment unit cooperatively form a circuit;
a laser emission unit, electrically connected to the connection interface unit to emit a laser, wherein the laser emission unit is fixed on the organism through the first electrically-conductive attachment unit and the second electrically-conductive attachment unit, and the laser emitted by the laser emission unit irradiates the organism; and
an open-circuit detection control unit, electrically connected to the first electrically-conductive attachment unit and the second electrically-conductive attachment unit, wherein
when the open-circuit detection control unit detects that the circuit is in an open-circuit state, the laser emission unit stops emitting the laser.

9. The laser emitter according to claim 8, wherein a power of the laser is between 3 milliwatts and 6 milliwatts.

10. The laser emitter according to claim 8, further comprising:
an emitting head, wherein the connection interface unit, the laser emission unit, and the open-circuit detection control unit are disposed at the emitting head, a first electrically-conductive connecting member and a second electrically-conductive connecting member are further disposed at the emitting head, and the first electrically-conductive connecting member and the second electrically-conductive connecting member are electrically connected to the open-circuit detection control unit; and
a bearing sheet body, wherein the first electrically-conductive attachment unit and the second electrically-conductive attachment unit are disposed on a side of the bearing sheet body, a third electrically-conductive connecting member and a fourth electrically-conductive connecting member are disposed on the other side of the bearing sheet body, and an opening is provided on the bearing sheet body, wherein
the third electrically-conductive connecting member is electrically connected to the first electrically-conductive attachment unit, and the third electrically-conductive connecting member is detachably connected to the first electrically-conductive connecting member,
the fourth electrically-conductive connecting member is electrically connected to the second electrically-conductive attachment unit, and the fourth electrically-conductive connecting member is detachably connected to the second electrically-conductive connecting member, and
the bearing sheet body is fixed to the emitting head through the third electrically-conductive connecting member and the fourth electrically-conductive connecting member, and the laser emitted by the laser emission unit irradiates the organism through the opening.
